(19) United States
(12) Patent Application Publication
(10) Pub. No.: US 2026/0165738 A1
(43) Pub. Date: Jun. 18, 2026

(54) MEDICAL INSTRUMENT FOR THE MULTIDIRECTIONAL ATOMISING OF A FLUID INTO A CAVITY OF A BODY, AND TOOL THEREFOR

(71) Applicant: REGER MEDIZINTECHNIK GMBH, Villingendorf (DE)

(72) Inventor: Alexander HETZEL, Villingendorf (DE)

(73) Assignee: REGER MEDIZINTECHNIK GMBH, Villingendorf (DE)

(21) Appl. No.: 19/125,345

(22) PCT Filed: Nov. 23, 2023
(86) PCT No.: PCT/EP2023/082840

§ 371 Date: Apr. 29, 2025

(30) Foreign Application Priority Data
Nov. 24, 2022 (DE) 10 2022 131 155.4

(51) Int. Cl. A61B 17/34; A61B 17/115; A61B 17/29; B05B 1/26

(52) U.S. Cl. CPC A61B 17/3462; A61B 17/1155; A61B 17/29; A61B 17/3423; B05B 1/26; A61B 2217/005

(57) ABSTRACT

A tool for a medical instrument for the multidirectional atomizing of a fluid into a cavity of a body has a shaft with a lumen. At the distal end thereof, a nozzle head is arranged, which has at least two lateral nozzles and one distal nozzle that is arranged on a distal end face of the nozzle head. Each nozzle has a nozzle opening that is fluidically connected to the lumen via a distributor cylinder. A cap is releasably arranged around the nozzle head, which cap has, for each of the nozzles, a through opening which can be positioned over their nozzle opening and which is designed to allow the discharge of a stream of the fluid to be atomized. The through openings of the cap each have an opening cross section that is smaller than a diameter of the respective nozzle positioned under the through opening.

### CROSS-REFERENCE TO RELATED APPLICATION

This application is a national stage application, filed under 35 U.S.C. § 371, of International Patent Application PCT/EP 2023/082840, filed on Nov. 23, 2023, which claims the benefit of German Patent Application DE 10 2022 131 155.4, filed on Nov. 24, 2022.

### TECHNICAL FIELD

The disclosure relates to a medical instrument for the multidirectional atomizing of a fluid into a cavity of a body and to a tool therefor.

### BACKGROUND

Tools for medical instruments, which are formed to bring fluids, in particular fluids with therapeutic substances, into a cavity of a body, are known from the prior art. WO 2012/163 346 A1 describes a trocar system with a tool, which has a nozzle on its distal end, wherein the distal end can protrude into a body cavity. A therapeutic fluid can be sprayed into a pneumoperitoneum by means of this nozzle.

The spraying direction of this nozzle, however, only points in one direction, so that the fluid cannot be sprayed multidirectionally within the abdominal cavity and thus evenly in all directions.

Tools with several nozzles are known from DE 10 2018 121 513 A1, wherein the tool head is rotatably mounted in the shaft of the tool there. The nozzle bodies are arranged at predetermined angles to one another in the spherical tool head, so that the nozzle openings are distributed evenly over the surface of the tool head.

DE 10 2018 121 496 A1 also relates to a medical instrument and a tool therefor, which serves for the directional introduction of a therapeutic substance into a cavity of a body. For this purpose, the tool has a shaft with a lumen with a flexible fluid line and a nozzle head. The flexible fluid line there extends in the lumen of the shaft and the nozzle head is connected thereto.

Lastly, U.S. 2013 / 325 059 A1 relates to a spray device, which is coupled to a dual syringe device, the outlets of which open out into many small holes with a size, which is suitable to allow liquid to escape at high speed.

U.S. Pat. No. 7,021,561 B2 further also describes a spray device with nozzles, which can atomize a spray of a medicament.

A syringe-shaped spraying device is further known from U.S. 2021/196 906 A1, which has a cylinder, a nozzle, a seal arranged therebetween.

### SUMMARY

It is an object of the present disclosure to provide an improved tool for a medical instrument for the multidirectional spraying or atomizing, respectively, of a fluid into a cavity of a body, which provides for a safe operation.

This object is solved by means of a tool as disclosed and claimed.

A further object of providing a medical instrument, which is formed for the multidirectional spraying or atomizing, respectively, of a fluid into a cavity of a body and which provides for the safe operation of the tool connected thereto, is solved by means of the medical instrument with the features as disclosed and claimed.

According to a first embodiment of a tool, which is used with a medical instrument in order to be able to multidirectionally atomize a fluid into a cavity of a body, the tool has a shaft with a lumen. A nozzle head, which has two or more lateral nozzles and one distal nozzle, is arranged on the distal end of the tool; the distal nozzle is arranged on a distal end face of the nozzle head. Each nozzle has a nozzle opening, which is fluidically connected to the lumen via a distributor cylinder. A cap is releasably arranged around the nozzle head thereby, which cap has, for each of the nozzles, a through opening, which is positioned over the nozzle opening thereof and which is formed to allow the discharge of a stream of the fluid to be atomized. The through openings of the cap each have a diameter, which is smaller than a diameter of the respective nozzle positioned under the through opening.

The nozzle head is further cylindrical and tapers over a shoulder on its end facing the shaft and forms a hollow-cylindrical receiving section. A pin is received in the lumen. A gap, which provides a fluid path for the fluid, is thereby formed between the outer circumference of the pin and the inner circumference of the shaft.

On its end facing the nozzle head, the pin has a section with a tapered outer circumference. While the hollow-cylindrical receiving section of the nozzle head extends into the shaft, the hollow-cylindrical receiving section surrounds the section with the tapered outer circumference of the pin.

Here, “cavity” of a body, into which a fluid is to be introduced, can be understood to be any type of cavity in a human or of an animal. Such a cavity can be the abdominal cavity, for example. Any fluid therapeutic substance, such as a medicament, medicament in solution or also a simple rinsing solution is possible as fluid, provided that it can be introduced into the cavity of the body by means of spraying and therefore “atomizing” in terms of dispensing by means of a nozzle. Here, “atomizing” thus refers to splitting up the fluid flowing in the tool into a large number of individual droplets by pressing through a nozzle and is to be equated with spraying or outputting via a nozzle or—depending on the used nozzle or nozzle opening, respectively—nebulizing. “Nozzle” is understood to be a component, which has a nozzle body (preferably cylindrical, but not limited thereto), in which at least one through bore or opening for a fluid to be atomized is formed.

The fluid to be introduced into the cavity is advantageously atomized multidirectionally through the distal nozzle and the lateral nozzles. The cap thereby advantageously provides a retention for the nozzle bodies of the nozzles and an additional security because the fluid can generate a high pressure in the tool lumen and can have an impact on the nozzle bodies of the nozzles in such a way that the nozzles are popped out of their anchoring in the nozzle head and, when being used in a patient, can injure said patient. The cap prevents this precisely in that it completely surrounds the nozzle body and in that the nozzle bodies are retained on the wall due to their larger dimensions, compared to the dimensions of the through openings of the cap.

The arrangement of the cap according to the disclosure thus advantageously offers a solution compared to the prior art, in the case of which the nozzle bodies are arranged in an unsecured manner in the tool head, and the fluid to be sprayed, which generates a high pressure within the tool, can for that very reason be popped out of the tool head and can injure a patient or the operator of the tool.

In the mounted state, the hollow-cylindrical receiving section extends into the shaft. Alternatively, the nozzle head can also have a different basic geometric shape, thus, e.g., elliptical. The pin thus extends into the nozzle head, wherein the pin extends in the proximal direction into the vicinity of a connecting piece of the shaft and thus creates a continuous gap, which ensures an even fluid flow through the shaft to the nozzle head. The receiving sections of the components pin and nozzle head can have fastenings. They can be threads or undercuts engaging with one another. The components can also be pressed or adhered to one another.

The through opening opens out into a recess in the jacket surface of the cap, which widens conically from the inside to the outside with respect to the jacket surface. This recess serves the purpose of guiding the fluid escaping from the nozzles. In a further embodiment of the tool, the through openings can also be smaller than the discharge openings of the nozzle bodies of the nozzles. When the through openings are smaller than the discharge openings, stronger forces, which are created in the nozzle bodies of the nozzles due to the compressed fluid, which flows through at high speed, can be guided, caught or distributed skillfully than when the through openings and the discharge openings are of equal size, and can thus further intensify the retaining function.

According to a further embodiment of the tool, the nozzle head has three, four or more lateral nozzles. The nozzles are directed in the radial direction, so that the fluid to be atomized can escape at a right angle to a tool longitudinal direction. The fluid to be atomized can hereby be sprayed particularly well multidirectionally into a cavity of a body. Preferred spraying angles of the nozzles lie in an angular range of 50° to 120°, an angle of 90° is spanned particularly preferably. A different angular range or multidirectional spraying, respectively, in predetermined directions can thus be attained for different applications.

According to yet a further embodiment of the tool, the lateral nozzles can further be arranged equidistantly from one another in the axial direction with respect to a central axis of the shaft. Advantageously for the even output of the fluid, the angles, about which the nozzles are spaced apart from one another with respect to the central axis, can be 120° in the case of three nozzles, accordingly 90° in the case of four nozzles. Other, non-equidistant angle combinations are also possible, for example when the atomization of the fluid is to take place so as to be directed in a certain direction, which can certainly comprise a large angle. The nozzles lie at the same level along the central axis of the shaft, but can also be present so as to be graded at different levels, so that the lateral nozzles can be arranged spirally along the longitudinal expansion of the nozzle head.

According to yet a further embodiment of the tool, the nozzle head has a distal section, which has a first fastening device. The cap accordingly has a second fastening device, which can be brought into engagement with the first fastening device, in order to simply fasten the cap to the nozzle head.

It is particularly advantageous when the nozzle head tapers on the distal section over a step-like shoulder, on which the first fastening device is arranged. The arrangement of the fastening device on the distal section offers the additional advantage that the cap can be attached to and held firmly on the nozzle head, whereby the nozzles can be protected even better against being popped out.

The first fastening device can be an external thread on the nozzle head, the second fastening device is then an internal thread on the cap, which can simply be screwed onto the nozzle head after mounting of the nozzle head to the shaft. The threaded connection can further also be sealed or adhered. The shoulder between nozzle head and distal section offers a further adhesive or sealing surface, respectively, and can serve as stop for the counter thread of the cap.

Alternatively, the cap can also be adhered to the nozzle head or the proximal edge of the cap can be welded or pressed to the nozzle head. Additional alternatives are further possible, such as a bayonet closure or a tongue and groove connection.

According to yet a further embodiment of the tool, the pin has a radial through bore close to its section with the tapered outer circumference. Here, “close to” means that the radial through bore is arranged adjacent to the section with the tapered outer circumference or at a distance therefrom, respectively, which is small compared to the total length of the pin. On the section with the tapered outer circumference, the pin further has a central-axial bore, the one end of which opens out into the nozzle head and the other end of which opens out into the radial through bore. The fluidic connection between the lumen and the distributor cylinder in the nozzle head is made possible hereby.

According to yet a further embodiment of the tool, the nozzles can be formed cylindrically. This allows constructing a structurally simple nozzle head, which likewise has cylindrical recesses for the nozzles, into which the nozzles are glued in, pressed, welded or also screwed.

According to yet a further embodiment of the tool, the shaft has, on its proximal end, a connection for the fluidic connection of the lumen to a fluid source, wherein the connection can be a Luer lock connection. With this, the tool can be connected to all possible hand grips or handles, respectively, for the use of the tool. Alternatively, the fluidic connection can be established by means of a hose, which is fastened directly to the proximal end of the shaft.

The nozzle head can be produced by means of a generative manufacturing method and can be matched exactly to the shaft. An outer diameter of the shaft can lie in a range of 1 mm to 30 mm; 8 mm to 10 mm are preferred. This dimension fits into existing trocar systems.

The disclosure further relates to a medical instrument for the introduction of fluids into a cavity of a body. The instrument can have a handle, a fluid source as well as a tool, wherein the tool can be releasably connected to the handle and can be fluidically connected to the fluid source. The tool according to the disclosure is used.

According to a further embodiment of the medical instrument, the handle can be connected to a trocar, so that the tool with the trocar can be used within a trocar system. The medical instrument can further be connected to the fluid source via a flexible fluid line, wherein the fluid source can be releasably connected to the tool and the tool can be inserted into the trocar of the medical instrument. By actuating corresponding actuating elements on the handle or on the trocar, respectively, the substance to be sprayed can be applied to the tool, in that a fluidic connection is established between the fluid source and the tool. The fluid substance reaches through the lumen to the nozzle body, is guided through the central bore of the nozzle body to the distributor cone and there via the supply lines to the nozzle openings of the nozzles. The substance escapes from the nozzle openings and is sprayed.

Further embodiments of the tool and of the medical instrument as well as some of the advantages, which are associated with these and further embodiments, become clear and easier to understand due to the following detailed description with reference to the accompanying figures. Objects or parts thereof, which are essential identical or similar, can be provided with the same reference numerals. The figures are only a schematic illustration of an embodiment of the invention.

### BRIEF DESCRIPTION OF THE DRAWINGS

FIG. 1 shows a perspective view of a medical instrument with tool for the multidirectional atomizing of a fluid into a cavity of a body.

FIG. 2 shows a longitudinal section through the tool.

FIG. 3 shows a perspective partial section through the nozzle head with cap.

FIG. 4 shows a longitudinal section through the nozzle head.

FIG. 5 shows a section transversely through the nozzles mounted in the nozzle head.

### DETAILED DESCRIPTION

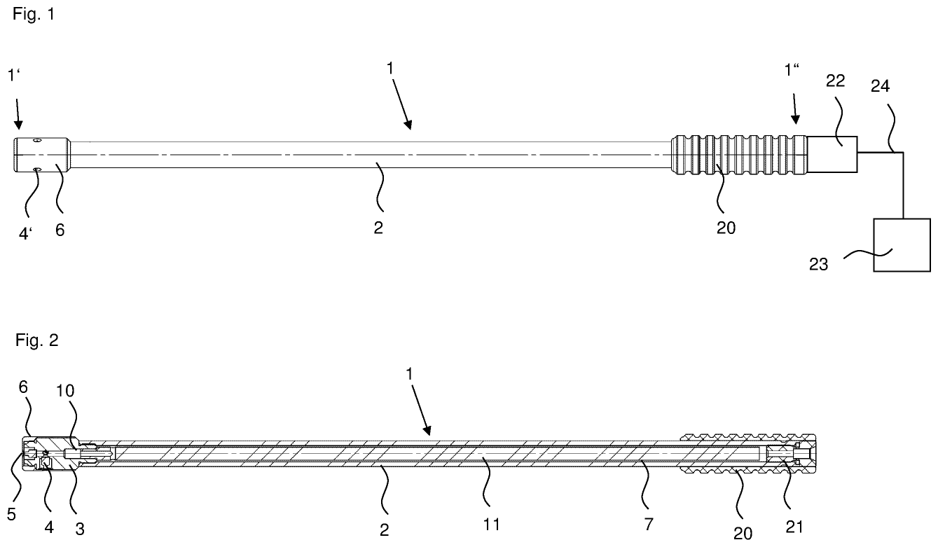

The medical instrument has a tool 1, which, in FIG. 1, has a shaft 2 with a proximal end 1″ and a distal end 1′ as well as an internal lumen 7. The shaft 2 is formed in an elongate and tubular manner and, in a preferred embodiment, has a diameter of approximately 10 mm. Diameters in a range of 1 mm to 30 mm are possible. A connecting piece 20, which has a so-called Luer lock connection 21, which provides a standard connection, to which different hoses or liquid accesses can be connected, is attached on the proximal end 1″. The Luer lock connection 21 is fluidically connected to the lumen 7 (see longitudinal sectional view in FIG. 2).

The tool 1 can be connected to a handle 22 and a fluid source 23 via the connecting piece 20, as shown in FIG. 1. The fluid source 23 is fluidically connected to the handle 22 via a fluid line 24 and thus to the tool 1. The tool 1 can further also be used together with a trocar system as part of the minimally invasive surgery (not illustrated figuratively).

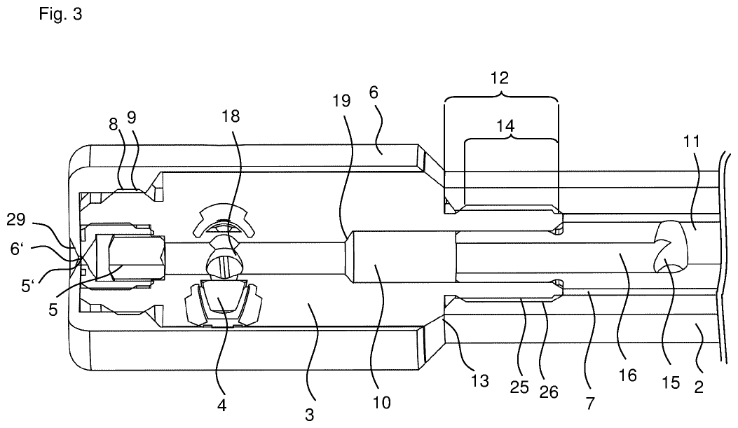

FIG. 2 to 5 show the tool 1 in individual details.

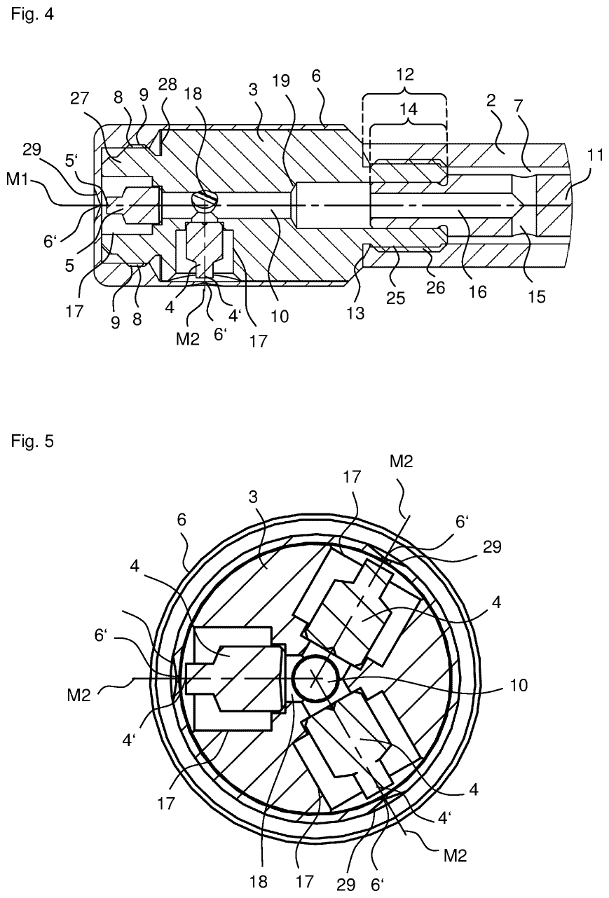

A cylindrical nozzle head 3 is arranged on the distal end 1′of the tool 1, which cylindrical nozzle head has a shoulder 13 on its end facing the shaft 2, via which shoulder the nozzle head 3 tapers and forms a hollow-cylindrical receiving section 12. The nozzle head is inserted in the shaft 2 with this receiving section 12. As shown in FIG. 4, the receiving section 12 has, on its jacket surface, an external thread 25, which engages with an internal thread 26 on the inner wall of the shaft 2. A section of a pin 11, which is received in the lumen 7 and which extends from the nozzle head 3 to just in front of the connecting piece 20 within the shaft 2, is inserted into the receiving section 12. A gap, through which a fluid can flow, is formed between an outer circumference of the pin 11 and an inner circumference of the shaft 2, as shown in FIG. 2 to 4. On its end facing the nozzle head 3, the pin 11 has a section 14 with a tapered outer circumference, with which the pin 11 is inserted into the hollow-cylindrical receiving section 12 of the nozzle head 3. The section 14 extends to just in front of the shoulder 13 and ensures that a continuous fluid path is formed for the fluid to be atomized in the shaft 2. So that the fluid is further guided out of the gap between pin 11 and inner wall of the shaft 2, the pin 11 has, adjacent to its section 14 with the tapered outer circumference, a radial through bore 15, which is fluidically connected to the gap in the lumen 7. In order to fluidically connect the nozzle head 3, the section 14 with the tapered outer circumference has a central-axial bore 16, the one end of which opens out into the nozzle head 3 and the other end of which opens out into the radial through bore 15.

A cylindrical distributor cylinder 10, which extends all the way to the distal end face of the nozzle head 3 starting at the receiving section 12, is introduced in the nozzle head 3, wherein the distributor cylinder 10 tapers on a step 19 in the direction of the distal end face of the nozzle head 3. On the one hand, bores 18 extend radially outwards from the distributor cylinder 10 in a plane, which is spanned by central axes M2 and, on the other hand, distally to the end face, and open out into cylindrical recesses 17, in which cylindrical nozzles 4, 5 are arranged. As a whole, the nozzle head 3 has three lateral nozzles 4 and one distal nozzle 5, which is arranged on the distal end face of the nozzle head 3 (according to the distal end of the tool 1′). The lateral nozzles 4 are spaced apart equidistantly from one another in the axial direction with respect to a central axis M1 of the shaft 2. According to the sectional drawing of FIG. 5, the central axes M2 of the lateral nozzles 4 each draw an angle of 120° with one another. Each nozzle 4, 5 has a nozzle opening 4′, 5′, which is fluidically connected to the lumen 7 via the distributor cylinder 10 and the bores 15, 16 in the pin 11. The recesses 17 are dimensioned so that the nozzle bodies of the nozzles 4, 5 can be received completely. They each have a grading, in which the nozzle bodies are inserted and fixedly mounted, as shown in FIG. 4. The “fixed mounting” can take place by means of gluing in, screwing or clamping.

A cap 6 is releasably arranged around the nozzle head 3. For each of the nozzles 4, 5, the cap 6 has a through opening 6′, which is positioned over the nozzle opening 4′, 5′thereof. These through openings 6′ introduced into the cap 6 allow a discharge of a stream of the fluid to be atomized. The dimensions of the through openings 6′ of the cap 6 each have an opening cross section, which is smaller than a diameter of the respective nozzle 4, 5, which is positioned under the through opening 6′. Each through opening 6′ widens into a conical recess 29, in order to guide the fluid escaping from the nozzles 4, 5. The diameter of the recess 29, just like the through opening 6′, is smaller than the diameter of the nozzle body of the nozzle 4, 5 arranged therebelow. The conical recess 29 distributes the forces, which act on the nozzle 4, 5 by means of the highly compressed fluid, which escapes through the through opening 6′.

To connect the cap 6 to the nozzle body, the nozzle head 3 has a distal section 27 on its jacket surface, which has an external thread 8, which serves as a first fastening device. The nozzle head 3 tapers on the distal section 27 via a step 28. The external thread 8 is formed on a section of the distal section 27. In a section corresponding to the distal section of the nozzle head 3, the cap 6 has, for this purpose, an internal thread 9, which is formed as counter thread to the external thread 8 and which serves as second fastening device. The cap 6 can thus be firmly screwed on. The step 28 on the nozzle head 3 forms a further surface for a possible seal or as stop for the counter thread 9 of the cap.

1.-12. (canceled)

13. A tool (1) for a medical instrument, configured for multidirectional atomization of a fluid into a cavity of a body, comprising:
a shaft (2) with a lumen (7);
a nozzle head (3) having nozzles (4, 5), including at least two lateral nozzles (4) and a distal nozzle (5),
wherein the nozzle head (3) is arranged on a distal end (1′) of the tool (1),
wherein the distal nozzle is arranged on a distal end face (3′) of the nozzle head (3),
wherein each nozzle has a nozzle opening (4′, 5′),
wherein each nozzle opening (4′, 5′) is fluidically connected to the lumen (7) via a distributor cylinder (10); and
a cap (6), the cap (6) being releasably arranged around the nozzle head (3),
wherein the cap has a respective through opening (6′) for each of the nozzles (4, 5),
wherein each respective through opening (6′) is positioned over the respective nozzle opening (4′, 5′) of the respective nozzle (4, 5) and formed to allow a discharge of a stream of the fluid to be atomized,
wherein each respective through opening (6′) has a diameter that is smaller than a diameter of the respective nozzle (4, 5) positioned under the respective through opening (6′),
wherein the nozzle head (3) is cylindrical and tapers over a shoulder (13) on an end facing the shaft (2) and forms a hollow-cylindrical receiving section (12), and
wherein a pin (11) is received in the lumen (7), and
wherein a gap provides a fluid path for the fluid between an outer circumference of the pin and an inner circumference of the shaft (2),
wherein, on an end facing the nozzle head (3), the pin (11) has a section (14) with a tapered outer circumference, and
wherein the hollow-cylindrical receiving section (12) of the nozzle head (3) extends into the shaft (2) and surrounds at least a portion of the section (14) with the tapered outer circumference of the pin (11).

14. The tool (1) according to claim 13,
wherein the nozzle head (3) has at least three lateral nozzles (4).

15. The tool (1) according to claim 13,
wherein the lateral nozzles (4) are arranged equidistantly from one another in an axial direction with respect to a central axis (M1) of the shaft (2).

16. The tool (1) according to claim 13,
wherein each respective through opening (6′) opens out into a recess in a jacket surface of the cap, and
wherein the jacket surface of the cap widens conically from an inside to an outside with respect to the jacket surface.

17. The tool (1) according to claim 13,
wherein the nozzle head (3) has a distal section (27) with a first fastening device,
wherein the cap (6) has a second fastening device, and
wherein the second fastening device can be brought into engagement with the first fastening device.

18. The tool (1) according to claim 17,
wherein the nozzle head (3) tapers on the distal section (27) over a step (28) and the first fastening device is arranged on the distal section (27).

19. The tool (1) according to claim 17,
wherein the first fastening device is an external thread (8) on the nozzle head (3) and the second fastening device is an internal thread (9) on the cap (6).

20. The tool (1) according to claim 13,
wherein the pin (11) has a radial through bore (15) close to its section (14) with the tapered outer circumference,
wherein the pin (11) has a central-axial bore (16) on the section (14) with the tapered outer circumference,
wherein one end of the central-axial bore (16) opens out into the nozzle head (3) and another end of the central-axial bore (16) opens out into the radial through bore (15).

21. The tool (1) according to claim 13,
wherein the nozzles (4, 5) are cylindrical.

22. A medical instrument for multidirectional atomization of a fluid into a cavity of a body, comprising:
a handle (22);
a fluid source (23); and
a tool configured to be releasably connected to the handle (22) and to be fluidically connected to the fluid source (23),
wherein the tool comprises
a shaft (2) with a lumen (7);
a nozzle head (3) having nozzles (4, 5), including at least two lateral nozzles (4) and a distal nozzle (5),
wherein the nozzle head (3) is arranged on a distal end (1′) of the tool (1),
wherein the distal nozzle is arranged on a distal end face (3′) of the nozzle head (3),
wherein each nozzle has a nozzle opening (4′, 5′),
wherein each nozzle opening (4′, 5′) is fluidically connected to the lumen (7) via a distributor cylinder (10); and
a cap (6), the cap (6) being releasably arranged around the nozzle head (3),
wherein the cap has a respective through opening (6′) for each of the nozzles (4, 5),
wherein each respective through opening (6′) is positioned over the respective nozzle opening (4′, 5′) of the respective nozzle (4, 5) and formed to allow a discharge of a stream of the fluid to be atomized,
wherein each respective through opening (6′) has a diameter that is smaller than a diameter of the respective nozzle (4, 5) positioned under the respective through opening (6′),
wherein the nozzle head (3) is cylindrical and tapers over a shoulder (13) on an end facing the shaft (2) and forms a hollow-cylindrical receiving section (12), and
wherein a pin (11) is received in the lumen (7), and
wherein a gap provides a fluid path for the fluid between an outer circumference of the pin and an inner circumference of the shaft (2),
wherein, on an end facing the nozzle head (3), the pin (11) has a section (14) with a tapered outer circumference, and
wherein the hollow-cylindrical receiving section (12) of the nozzle head (3) extends into the shaft (2) and surrounds at least a portion of the section (14) with the tapered outer circumference of the pin (11).
